(19) United States
(12) Patent Application Publication
(10) Pub. No.: US 2001/0034062 A1
(43) Pub. Date: Oct. 25, 2001

(54) Antibody gene therapy with adeno-associated viral vectors

(76) Inventor: Scott Koenig, Rockville, MD (US)

Correspondence Address: CARELLA, BYRNE, BAIN, GILFILLAN, CECCHI,, STEWART & OLSTEIN, 6 BECKER FARM ROAD, ROSELAND, NJ, 07068, US

(21) Appl. No.: 09/781,052

(22) Filed: Feb. 9, 2001

Related U.S. Application Data
(60) Provisional application No. 60/182,312, filed on Feb. 9, 2000.

(51) Int. Cl. A61K 48/00; C12N 15/861; C12N 5/10

(52) U.S. Cl. 435/456; 424/93.21; 435/325; 435/320.1

(57) ABSTRACT

Methods are disclosed for treating and/or preventing diseases, both infectious and chronic. Infectious diseases combated by the methods disclosed herein include microbial caused diseases, especially diseases of the respiratory system, including those diseases caused, induced or otherwise mediated by viruses, bacteria, fungi and other parasites. The present invention also discloses vectors useful in the treatment and/or prevention of such conditions. The vectors disclosed herein are recombinant viral vectors comprising genetically engineered adeno-associated viruses comprising genetic sequences coding for antibody molecules and portions thereof, which antibodies are useful in combating or preventing infectious and chronic diseases, such as respiratory diseases, including asthma, as well as ophthalmic diseases, including age related and diabetes-related macular degeneration and other retinopathies.

This application claims priority of U.S. Provisional Application Ser. No. 60/182312, filed Feb. 9, 2000, the disclosure of which is hereby incorporated by reference in its entirety, as are all other cited references relied on herein.

### FIELD OF THE INVENTION

The present invention is directed to a method of delivering antibody-encoding DNA through the use of viral vectors, especially recombinant adeno-associated viruses, and the use of viral vectors, such as adeno-associated vectors, in engineering cells for delivery of antibodies for the purpose of combating disease, including respiratory diseases and diseases affecting other organs and systems.

### BACKGROUND OF THE INVENTION

Antibodies have been, and are currently being, developed for the prevention and treatment of various diseases.

Among the more difficult infectious agents to control and treat are the viruses. For example, respiratory syncytial virus (RSV) is a major cause of acute respiratory illness in young children admitted to hospitals and the major cause of lower respiratory tract infection in young children. Efforts to produce a vaccine against such viruses as RSV have proven unsuccessful. Other viruses involved in respiratory diseases have been similarly resistant, if not intractable. Conversely, efforts to produce antibodies useful in controlling such viruses have resulted in preparation of antibodies having extremely high affinities. Such antibodies are patented and currently available commercially. [See: U.S. Pat. No. 5,824,307, the disclosure of which is hereby incorporated by reference in its entirety].

Efforts to produce such antibodies have often involved methodologies where chimeric or hybrid antibodies are produced. In such cases, the antibody molecules will often have polypeptide regions selected from different sources, including different species of animals. For example, variable regions of an antibody of one species have been successfully grafted onto the constant region of an antibody of a different species. In addition, sometimes only the hypervariable regions of an antibody, such as a murine antibody specifically selected for affinity for the antigen of interest, such as RSV or some other respiratory disease causing virus, or other microbe, can be grafted onto the constant and framework regions of a human antibody, thereby providing high specificity and affinity for the antigen of interest while preventing a secondary immune response against the antibody being used. Such “humanized” antibodies have proven highly successful. Further, because of the advent of methods of molecular biology and recombinant DNA technology, it is now possible to prepare antibody molecules, both heavy and light chain polypeptides, from the corresponding polynucleotide sequences, which sequences may be introduced into specified cell lines for synthesis, assembly and secretion of the expressed proteins by said cells. Thus, almost any desired antibody molecules can be prepared once the polypeptide sequences are known for the respective heavy and light chains. In addition, selected amino acid residues thereof can be specifically altered, or mutated, or otherwise replaced by more advantageous amino acids, in order to optimize the biological activity of the antibody.

However, regardless of the affinity and biological activity of the antibody produced, a critical concern in clinical use of antibodies is the method of delivery. Often, antibody activities (i.e., potencies or biological activities) can be relatively low (often on the order of tens of milligrams per kilogram of body weight) thereby requiring large doses be given intravenously or by some other means (depending on the physical state of the antibody preparation, be it a powder, a suspension, or the like). In addition, the cost of in vitro production of such antibody preparations can have a limiting effect on the availability and utility of such antibody preparations, regardless of how clinically effective they may be. It would therefor be highly useful and advantageous to have available a means of providing a continuous measured amount of antibody, and at a relatively low cost, for use in the treatment of infections, especially respiratory infections, such as RSV or even chronic diseases such as asthma. In a similar way, other antibodies are useful against other types of disorder. For example, antibodies useful for treating age related ophthalmic disorders, including diabetes-related macular degeneration and diabetes-related retinopathies. Such antibodies are highly useful within the methods of the present invention as disclosed herein.

One promising route of administration for treatment of disease is gene delivery to selected cells. By such a procedure, cells can be altered recombinantly to have inserted therein one or more genes of interest, such as genes coding for the heavy and light chains of an antibody, especially an antibody useful against a microbe, such as a virus or other pathogens, including respiratory microbes such as bacteria, fungi and parasites. Such methods are also useful in regard to other diseases via delivery of other types of antibodies, especially those useful in the treatment of different types of ophthalmic disease. Such cells, e.g., muscle and respiratory cells, can then be inserted into the tissues of a given organ of the recipient patient wherein the cells will express the exogenous DNA, and synthesize and secrete the antibody protein into the spaces surrounding the cells for delivery to the blood stream or local tissues, depending on where the antibody is to realize its clinical effects. Such a means of administration allows the inserted engineered cells to produce constant, possibly even inducible, levels of antibody protein without the commercial costs and problems of producing large lots of antibody that must then be stored until use. In addition, the cells can be engineered to respond to the needs of the patient in producing varying amounts of the antibody protein as required.

While such a route of administration is attractive from both a theoretical and practical point of view, the selection of the cells to engineer, as well as the vector to be used, must be carefully considered. Not all cells are capable of being engineered to produce the desired proteins. In addition, fully functional antibodies are polypeptide tetramers with 2 light and 2 heavy chains. Even where Fab or other immunologically active fragments are used, two chains are required. The polypeptide chains must be properly joined together, for example by disulfide bonds, and the antibody protein itself may require a certain degree of glycosylation in order to have its normal biological effects. Thus, any engineered cells may have to be recombinantly modified to produce not only the antibody protein but also ancillary proteins, such as enzymes, required to suitably modify the antibody protein once synthesized. In the case of antibodies useful in the present invention, cellular candidates for genetic engineering may include muscle cells, for intramuscular delivery to the bloodstream or to localized areas of infection, as well as respiratory cells, for more or less direct delivery to the site of a respiratory infection, or for administration to the visual system for treatment of ophthalmic conditions, including various retinopathies. Such methods of delivery are also useful in combating other types of disease process, both chronic and infectious.

As used herein, and unless expressly stated to be otherwise, the term “antibody” is to be understood as synonymous with the term “immunoglobulin” and both terms are understood to include variations of such molecules, including portions, fragments, and segments thereof, so long as the latter retain sufficient immunological activity so as to achieve substantially the same therapeutic effect as the whole antibody.

Among the vectors useful for such purposes has been the adenoviruses, useful for a wide range of host cell types, although the latter have had the drawback of producing proteins that can elicit unwanted immunological responses, a result distinctly limiting their effectiveness in gene therapies.

Alternatively, use of adeno-associated viruses (AAV) has proven of value for gene delivery methods. These viruses possess a linear, single-stranded DNA genome of about 4,700 nucleotides in length with terminal inverted repeats of some 150 bases in length at each end to function as origins of replication. Further, in vitro packaging of adeno-associated virus DNA has been accomplished. [See: U.S. Pat. No. 5,741,683—the disclosure of which is incorporated by reference]

AAV replication usually requires infection with an unrelated helper virus, which can include such viruses as adenovirus, herpesvirus or vaccinia. Such helper viruses serve to facilitate a productive infection by supplying accessory genes whose expression is necessary for many steps in the AAV replication process. In the absence of such infection, AAV can establish a latent state by integration of its DNA into a host cell chromosome. If the cell is later infected by any of the aforementioned helper viruses, such later infection serves to “rescue” the AAV-integrated DNA thereby facilitating replication of the integrated AAV to produce new infectious virus particles. AAV has an almost unlimited host cell range among mammalian cell targets so long as a helper virus is available or the AAV particles are suitably engineered to be infectious without the helper viruses. In addition, AAV appears to be associated with no known human diseases, so problems of potential infection by the vector are thus avoided (as opposed to adenovirus). In this way, selected gene sequences can be cloned within the resulting recombinant AAV vectors or plasmids and thence used for therapeutic purposes as disclosed herein. The functioning of this genome is well known in the literature. (See: Muzyczka, N. (1992) Current Topics in Microbiol. and Immunol. 158:97-129 for a review). Methods of preparing recombinant AAV vectors or plasmids are well known in the art. [See: U.S. Pat. Nos. 5,173,414 and 5,139,941; International Publication Numbers WO 92/01070 (published Jan. 23, 1992) and WO 93/03769 (published Mar. 4, 1993) and Kotin, R. M. (1994) Human Gene Therapy 5:793-801.

The production of recombinant AAV is readily achieved through the use of an AAV vector, such as an AAV plasmid, that is cotransfected into a host cell along with an ancillary DNA construct capable of providing the helper functions normally required for AAV replication. The AAV vector normally comprises the inserted DNA, such as that coding for an antibody, flanked by the aforementioned terminal repeats. This is co-transfected with the accessory DNA, such as a helper plasmid, that contains rep and cap coding regions, well known in the art, but lacking the terminal repeats so that this latter structure, or plasmid, cannot replicate or package itself. The rep or cap coding regions provide the promoters for replication of the AAV vector and are commonly activated in a trans fashion using either subsequent infection with a helper virus (as mentioned above) or using a vector engineered to provide such helper functions. The AAV viruses comprising the exogenous DNA are then produced on subsequent culturing of the transformed cells and may be recovered from the culture medium. These recombinant AAVs are then available for gene transfer into selected tissues and cells.

Recombinant AAV viruses (rAAV) have been shown to be useful for infection of respiratory epithelial cells (Flotte et al. (1992) Am. J. Respir. Cell Mol. Biol. 7:349-356; Flotte et al. (1993) J. Biol. Chem. 268:3781-3790; Flotte et al. (1993) Proc. Natl. Acad. Sci. USA 90:10613-10617) and for transformation of nonproliferating cells (See: Flotte et al. (1994) Am. J. Respir. CellMol. Biol. 11:517-521).

In accordance with the present invention, rAAV finds use in applications whereby the rAAV is used as a vector for delivery of the polynucleotides coding for antibody polypeptides to cells useful in the methods disclosed herein, such as muscle and respiratory cells. Adeno-associated virus is especially useful for inducing the expression of anti-RSV antibodies, such as an antibody having the same specificity as that of an antibody as disclosed in U.S. Pat. No. 5,824,307, because AAV can itself induce expression of foreign genes to as much as 1 mg/ml in serum whereas expression of as little as 0.1 mg/ml of antibodies disclosed herein is ordinarily sufficient to prevent lung infection, such as infection by respiratory syncytial virus (RSV), as well as other microbes causing respiratory distress, including other viruses, as well as bacteria, fungi and parasites infecting the respiratory tract, and perhaps even prevent nasal replication of such organisms. The antibodies disclosed as useful within the methods of the present invention are also available to combat chronic diseases, such as asthma and various ophthalmic diseases, such as macular degeneration, especially that associated with diabetes or progressing age. Of course, concentrations in local areas are expected to be higher and therefor the antibody will be even more effective in dealing with local infections.

For the methods of the present invention, muscle cells are a useful gene delivery target, in addition to respiratory tissue, being readily accessible, well-differentiated and non-dividing [See: Barr and Leiden (1991) Science 254:1507-1509], properties important for optimal gene transfer. Delivery of genes to muscle has been demonstrated in the literature, along with subsequent systemic appearance of proteins encoded by the exogenous genes of the AAV vectors. [See, for example, U.S. Pat. No. 5,858,351, and references cited therein]

### BRIEF SUMMARY OF THE INVENTION

The present invention relates to methods for treating or preventing infections, such as respiratory infections, most especially viral induced respiratory infections, as well as chronic infections, such as asthma in its various forms, comprising administering to a patient in need thereof, or at risk thereof (in the case of an otherwise healthy individual), of a therapeutically effective amount of a recombinant adeno-associated virus (AAV or rAAV) comprising an exogenous polynucleotide encoding a polypeptide sequence of the light and/or heavy chain of an antibody effective against said respiratory viral infection. Such encoded antibodies may be tetramers, dimers, single chain antibodies, bifunctional antibodies, chimeric antibodies, humanized antibodies, wholly novel recombinant antibodies, antibodies synthesized de novo by chemical or biological means, Fab fragments, F(ab)₂′, and other immunoactive portions, fragments, segments and other smaller or larger partial antibody structures wherein the latter possess sufficient immunological stimulatory activity so as to be therapeutically useful within the methods of the present invention.

In other embodiments, the present invention provides antibody-encoding vectors for use in delivering antibody-encoding genes to cells, such as muscle, respiratory, and other cell types, in vitro or in vivo, whereby the antibodies, once expressed and released by the cells so transformed, are readily available for the treatment of respiratory diseases and diseases of the ophthalmic system. Such rAAV vectors are advantageously administered either by direct application to local tissues that then directly express the antibodies, often as needed in response to a disease process, or as a steady background production of such antibodies, or by remote production of said expressed antibodies and subsequent deposit into the bloodstream for transit to locations where disease processes are at work.

In some embodiments, this includes the use of a spectrum of antibodies for combating either infectious or chronic disease processes, including conditions such as asthma and macular degeneration, to name only a few.

It is also an object of the present invention to provide recombinant vectors, especially adeno-associated virus (AAV) vectors, genetically engineered to contain polynucleotides encoding polypeptides comprising the light and/or heavy chains, or both chains, of an antibody molecule effective in combating chronic respiratory conditions, such as asthma and the like. Such polynucleotides coding for different chains of said antibody molecules may be physically linked to each other or not, or may be separate polynucleotides present at different locations within the vector DNA or in separate rAAV vectors. Among the antibodies useful in such vectors are anti-Interleukin 9 (anti-IL-9) and anti-IL-9-receptor (an antibody specific for the cellular receptor that binds IL-9).

In a highly specific embodiment, the present invention relates to recombinant AAV vectors, including rAAV plasmids and the like) comprising exogenous DNA encoding at least one heavy or one light chain, or both, of an antibody specific for at least one epitope of RSV, preferably the F antigen or the G antigen of RSV, especially where said antibody is an antibody disclosed in U.S. Pat. No. 5,824,307.

It is a further object of the present invention to provide a recombinant cell comprising exogenous DNA wherein said DNA is at least 95% identical to a DNA encoding an antibody specific for an epitope of RSV, especially the F protein of RSV. Said cell will commonly be either a muscle cell, or a cell from the respiratory system, such as an epithelial or connective tissue cell. Such cell may be generated in vitro or in vivo using the recombinant vectors disclosed herein.

In a separate embodiment, the present invention also relates to a composition comprising a recombinant AAV vector suspended in a pharmacologically acceptable carrier, which may include any pharmaceutically useable diluent or excipient. Such recombinant AAV vectors may even be administered as an aerosol or by other delivery means.

The present invention also relates to methods of treating or preventing disease conditions, including both infectious and chronic diseases, by administering compositions containing the recombinant AAV vectors disclosed herein.

### DETAILED DESCRIPTION OF THE INVENTION

In its broadest terms, the present invention is directed to providing recombinant adeno-associated viruses whose genomes comprise an exogenous polynucleotide encoding a polypeptide sequence of the light and/or heavy chain of an antibody effective against said respiratory viral infection, so long as a dimeric or tetrameric structure with immunological activity is produced. The present invention also relates to methods for treating or preventing respiratory viral infections comprising administering to a patient in need thereof, or at risk thereof, of a therapeutically effective amount of said recombinant adeno-associated virus (AAV) vectors.

In accordance with one embodiment of the present invention, infections, especially respiratory infections, most especially virus infections of the respiratory system, are treated by administration to a patient, especially a child, or possibly an elderly person, of a therapeutically effective amount of recombinant AAV vectors, possibly as a powder or other type of aerosol, that has been genetically engineered to incorporate within its genome an exogenous polynucleotide encoding one or more polypeptides, including heavy and/or light chains, of an antibody that, when expressed, is therapeutically effective in combating infections caused by said respiratory disease causing virus, especially viruses causing respiratory distress, as well as non-viral microbes inhabiting, and infecting, the respiratory system, including bacteria, fungi and other respiratory parasites. Thus, an antibody encoded by the exogenous polynucleotide incorporated into the genome of said AAV vector is most advantageously an anti-viral antibody.

In a most specific embodiment, the antibody encoded by said exogenous polynucleotides is most advantageously an antibody specific for the F protein of RSV, such as, but not limited to, an antibody as disclosed in U.S. Pat. No. 5,824,307, the disclosure of which is hereby incorporated by reference

In addition, the present invention provides antibody-encoding vectors for use in delivering antibody-encoding genes to cells, such as muscle, respiratory, and other cell types, in vitro or in vivo, whereby the antibodies, once expressed and released by the cells so transformed, are readily available for the treatment of respiratory diseases and diseases of the ophthalmic system, either by direct application to local tissues that then directly express the antibodies, often as needed in response to a disease process, or as a steady background production of such antibodies, or by remote production of said expressed antibodies and subsequent deposit into the bloodstream for transit to locations where disease process are at work.

In some embodiments, this includes the use of a spectrum of antibodies for combating either infectious or chronic disease processes, including conditions such as asthma and macular degeneration, to name only a few.

Some of the antibodies effective against viruses are so-called “humanized” antibodies, so that some variation in the identity of the framework and/or complementarity determining regions (i.e., the CDRs or hypervariable regions of the heavy and light chain variable regions of said antibodies that are critical to determining the antigenic specificity of the antibodies) is both permitted and expected within the methods disclosed herein. Thus, in accordance with the present invention, the amino acid sequence(s) of the polypeptides of the antibodies useful for the present invention may show some variation from those given for antibodies of known utility against microbes and for use in treating chronic diseases. Such variations may include sequence homologies that are as much as 5%, thereby having at least a 95% identity with the antibody sequences useful herein. For example, such variations represent an amount that will still facilitate high specificity and affinity for epitopes found on viruses causing respiratory disease, such as RSV, especially the F epitope thereof, and specific epitopes located on viruses other than RSV, especially where such are respiratory disease-causing microbes, including other viruses, bacteria, fungi and other parasites. Thus, antibodies encoded by polypeptides inserted into the AAV vectors of the present invention can vary in amino acid sequence from the corresponding canonical antibodies and still be useful.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100[1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

Thus, any antibodies useful in treating or preventing diseases as disclosed herein are useful with the vectors of the present invention so that the recombinant vectors of the invention are not limited by the native or modified sequences of the antibodies.

Also in accordance with the present invention, the methods disclosed herein are equally effective in preventing diseases caused by viruses, especially respiratory diseases, but also including respiratory diseases caused by other microbes, such as bacteria, fungi and other parasites, and also chronic respiratory conditions such as asthma. The methods disclosed herein are also effective in dealing with diseases occurring elsewhere in the body, such as, for example, ophthalmic diseases, such as age related ophthalmic conditions, infections of the ophthalmic system, and non-infectious conditions, such as diabetes related macular degeneration.

In carrying out such methods in a patient not yet infected with a disease-causing organism, the patient would be treated in substantially the same way as a patient afflicted with a common respiratory disease, such as one caused by RSV or other pathogen, except that said patient would present in an apparently non-infected condition (i.e., a condition where viral infection of the type intended to be prevented by the methods disclosed herein are not evident from the usual testing scheme used to diagnose such conditions). In keeping with the present invention, therefore, such patient would be given a sufficient amount of the recombinant AAV (rAAV) according to the present invention so as to prevent development of a disease (where infection has occurred but is not readily diagnosable) or where no infection has yet occurred but such infection is believed at least possible, if not probably, and such infection is desired to be prevented from occurring.

In administering to a patient a therapeutically effective amount of the vectors disclosed herein, either for purposes of treatment of an active infection or for purposes of preventing infection, or preventing more serious infection, recombinant AAVs of the present invention will commonly be administered suspended in some type of medium. One embodiment of the present invention relates to a composition comprising a recombinant AAV vector, prepared according to the disclosure herein, suspended in a pharmacologically acceptable carrier, which can include any pharmaceutically acceptable diluent or excipient.

Such compositions will commonly be of sufficient concentration, or possess sufficient biological activity, or potency, that administration of such composition to a patient in need thereof will result in infection of cells of said patient, and subsequent expression of the exogenous DNA contained in said AAV vectors, that the resulting transformed cell or cells, or recombinant or genetically engineered cells resulting from such infection, will secrete into the tissues and/or bloodstream of the recipient of a therapeutically effective amount of the antibody encoded by said exogenous DNA as to effectively combat the virus-induced respiratory disease intended to be treated, or be sufficiently potent to prevent any subsequent infection by said virus, especially where said virus is a virus exhibiting strong surface antigens that readily attract circulating, or localized, antibodies, such as those produced by cells containing antibody-encoding genes like the transferred by the rAAV vectors of the present invention.

Aerosols are also useful for local or topical administration of the therapeutically active vectors of the present invention. Implanting of cells transformed by the recombinant AAV vectors disclosed herein are also a convenient mode of introducing therapeutic levels of the antibodies useful within the present invention, which can include most types of antibodies.

One difficulty with rAAV for gene transfer has been contamination with helper virus genes present during growth of stock suspensions of the rAAV particles (produced during cloning) as well as of the wild type AAV (which contains no exogenous DNA of interest). Consequently, alternative strategies utilizing no helper virus and instead employing in vitro packaging methods to produce the recombinant vectors are available from the patent literature. [See: U.S. Pat. No. 5,741,683 and methods disclosed therein] Recombinant AAV (rAAV) virions, particles or plasmids are known and can readily be prepared [See: U.S. Pat. Nos. 5,173,414 and 5,139,941; International Publication Numbers WO 92/01070 (published Jan. 23, 1992) and WO 93/03769 (published Mar. 4 1993) and Kotin, R. M. (1994) Human Gene Therapy 5:793-801] . As used herein, the term “coding region” refers to that portion of a gene which either naturally or normally codes for the expression product of that gene in its natural genomic environment, i.e., the region coding in vivo for the native expression product of the gene. The coding region can be from a normal, mutated or altered gene, or can even be from a DNA sequence, or gene, wholly synthesized in the laboratory using methods well known to those of skill in the art of DNA synthesis.

The term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon. Thus, a “polynucleotide” is simply a discrete and defined nucleotide sequence.

In forming the DNA constructs for coding for the immunoglobulin polypeptides according to the present invention, forward and reverse primers can readily be constructed from the known sequences of the antibodies selected for use and then employed in amplification procedures such as the polymerase chain reaction (PCR) or rolling circle polymerization (RCA).

As used herein, the term “expression product” means that polypeptide or protein that is the natural translation product of the gene and any nucleic acid sequence coding equivalents resulting from genetic code degeneracy and thus coding for the same amino acid(s). Thus, cells expressing a polynucleotide, or expressing the product encoded by said polynucleotide, are cells that make the polypeptide and which may or may not secrete such polypeptide into the surrounding medium or tissue spaces.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotides, such terms refer to the products produced by treatment of said polynucleotides with any of the common endonucleases.

As used herein, the term “fragment,” when referring to a coding sequence, means a portion of DNA comprising less than the complete coding region whose expression product retains essentially the same biological function or activity as the expression product of the complete coding region. Such regions may include genetic control elements essential for regulation of transcription.

As used herein, the term “primer” means a short nucleic acid sequence that is paired with one strand of DNA and provides a free 3′ OH end at which a DNA polymerase starts synthesis of a deoxyribonucleotide chain.

As used herein, the term “promoter” means a region of DNA involved in binding of RNA polymerase to initiate transcription.

As used herein, the term “open reading frame (ORF)” means a series of triplets coding for amino acids without any termination codons and is a sequence (potentially) translatable into protein.

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

As used herein, the term “helper virus” refers to a virus such as adenovirus, herpesvirus, cytomegalovirus, Epstein-Barr virus, or vaccinia virus, which when infected into an appropriate eukaryotic cell, allows a productive AAV infection to occur.

As used herein, the term “rAAV” refers to a recombinant AAV-DNA molecule containing some AAV sequences, usually at a minimum the inverted terminal repeats and some foreign or exogenous (i.e., non-AAV) DNA.

As used herein, the terms “AAV vector” or “AAV particle” or “AAV plasmid” refer to any adeno-associated virus vector or any vector derived from an adeno-associated virus. These include, but are in no way limited to, AAV-1, AAV-2, AAV-3, AAV-4, AAV-5, AAVX7, etc. Such AAV vectors or particles or plasmids may have one or more of the AAV wild-type genes deleted in whole or in part, preferably the rep and/or cap genes, but retain functional flanking terminal repeat sequences, the latter being necessary for the rescue, replication and packaging of the rAAV particle containing the exogenous DNA sequence of interest which codes for the desired immunoglobulin molecule. Therefore, rAAV vectors or particles within the present invention include the minimal sequences required in cis for replication and packaging of the vectors. These terminal repeat sequences may be any sequences having sufficient sequence homology to the wild-type sequences so that they can support replication of the recombinant viral vectors useful in the present invention (i.e., provide for functional rescue, replication and packaging during the preparation of the therapeutically useful vector particles). In accordance with the present invention, such vectors, particles or plasmids include any infectious particle produced by in vivo or in vitro packaging of DNA into an rAAV particle.

For purposes of in vitro packaging, the packaged DNA may comprise a reporter or marker gene, such as the inducible LacZ coding sequence, and may be under control of some pre-selected promoter sequence, for example, the CMV (cytomegalovirus) promoter).

As used herein, the term “transformation” refers to the transfer of a gene, or genes, to a cell by means of a vector, especially a recombinant vector, and most especially a virus particle, such that the gene is expressed in the cell.

As used herein, the term “gene delivery” refers to methods for inserting foreign, i.e., exogenous, DNA into host cells, especially into muscle or respiratory cells, using the methods of the present invention involving rAAV particles or vectors. Such methods may produce either transient or long term gene expression of exogenous DNA, extra-chromosomal replication and expression of transferred genes, or sequences for expression, but may also include expression where the exogenous DNA has become integrated into the genome of the target cells. Well known gene transfer systems available for mammalian cells include those described in U.S. Pat. Nos. 5,399,346 and 5,858,351.

As used herein, the term “vector” refers to any type of genetic element, such as a plasmid, phage, transposon, cosmid, chromosome, virus, etc., that is capable of replication when provided with appropriate control and accessory elements and which can transfer exogenous gene sequences into cells, including all manner of cloning and expression vehicles, as well as viral vectors, especially the rAAV vectors utilized in the present invention.

In accordance with the present invention, there are provided herein “recombinant AAV virions” (rAAV virions), which are infectious, replication-defective virus particles formed of an AAV protein shell surrounding an exogenous DNA molecule of interest which is flanked on both sides by AAV inverted terminal repeats. Such particles are produced in a suitable host cell which has had an AAV vector, AAV helper functions and accessory functions introduced therein and wherein the host cell is thereby capable of producing active rAAV particles for subsequent gene delivery into susceptible cells and tissues, especially the muscle and respiratory cells and tissues useful in practicing the invention as disclosed herein.

The term “transfection” is used to refer to the uptake of foreign DNA by a mammalian cell. A cell has been “transfected” when exogenous DNA has been introduced inside the cell membrane. A number of transfection techniques are known in the art. The term refers to both stable and transient uptake of the genetic material.

By “muscle cell” or “muscle tissue” is meant a cell or group of cells derived from muscle of any kind, including skeletal, smooth and cardiac, and excised from any area of the body. Muscle tissue associated with the respiratory tract or system is especially preferred. Such muscle cells may be differentiated or undifferentiated, such as myoblasts, and still find use within the present invention. Since muscle tissue is readily accessible to the circulatory system, a protein produced and secreted by muscle cells and tissue in vivo will logically enter the bloodstream for systemic delivery, thereby providing sustained, therapeutic levels of protein secretion from muscle.

By respiratory cell is meant any connective tissue cell or epithelial tissue cell found in the respiratory system or tract and may include both quiescent and actively proliferating cells. Because the methods disclosed according to the present invention seek to control respiratory-related diseases, production of antibodies specific for respiratory pathogens directly from cells of respiratory tissue has the advantage of immediate and sustained delivery of such therapeutic neutralizing antibodies to the area in which they will be most effective, without having to go through the circulatory system as an intermediate and thereby possibly reducing the dose and effectiveness of such agents.

Such respiratory or muscle cells may be cells found in the respiratory system or tract, or elsewhere in the case of muscle cells, including cells excised from said respiratory system or tract, or cells excised therefrom and then reinserted into the respiratory system or tract, or other tissues or organs in the case of muscle cells, including cells whose genomes have been recombinantly manipulated either in situ or in vitro so as to contain one or more exogenous, or heterologous, DNA or RNA sequences, inserted by use of the vectors disclosed herein, or otherwise, and which cells actively express, or, under suitable conditions, are capable of expressing polypeptides, especially antibody molecules, including immunologically active fragments, segments, or portions thereof, which antibody molecules are encoded by said exogenous, or heterologous, DNA or RNA sequences.

For purposes of the present invention, the terms “heterologous” and “exogenous” are deemed synonymous as they relate to DNA. Thus, this term refers most preferably to DNA that is to be inserted into a vector in accordance with the invention disclosed herein and which DNA is subsequently expressed by cells into whose genome the exogenous or heterologous DNA is inserted as a result of gene transfer using the rAAV vectors of the invention. Such DNA will commonly encode, or comprise sequences that encode, one or more polypeptide chains possessing antibody activity and specificity toward a disease causing microbial agent, especially a virus, and most especially where said virus induces or mediates, or otherwise causes, diseases of the respiratory system. In a preferred embodiment, such disease is caused by RSV and the antibody, or antibodies, or active fragments thereof, are specific for the F or G antigen of RSV. In specific embodiments, said exogenous or heterologous DNA encodes antibodies, or immunologically active fragments, segments, or portions thereof, with specificity for a virus or other infectious microbial agent. Preferred embodiments include exogenous or heterologous DNA sequences, and active fragments thereof, encoding one or more of the antibodies disclosed in U.S. Pat. No. 5,824,307.

As used herein, a “gene” or “coding sequence” or a sequence which “encodes” a particular protein, is a nucleic acid molecule which is transcribed (in the case of DNA) and translated (in the case of mRNA) into a polypeptide in vitro or in vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “control element” refers collectively to promoter regions, transcription termination sequences, upstream regulatory domains, origins of replication, enhancers, and the like, which collectively provide for the replication, transcription and translation of a coding sequence in a recipient cell. Not all of these control elements need be present in the same DNA or RNA sequence so long as the selected coding sequence is capable of being replicated, transcribed and translated in an appropriate host cell.

As used herein, the term “operably linked” refers to an arrangement of genetic elements, including control elements, such that when these elements are present within the same DNA sequence, or within the same exogenous or heterologous DNA sequence within a rAAV vector as described herein, or are otherwise arranged in cis with each other, they have their expected and natural biological function. For example, control elements operably linked to a coding sequence result in the expression of said coding sequence when conditions conducive to expression of said sequence are produced. Such control elements need not be contiguous within the coding sequence, but may be present in cis, or even in trans, provided that they direct the expression of said coding sequence when conditions are arranged so as to induce such expression. Intermediate sequences may be present yet the control elements may still be considered operably linked within the meaning and spirit of the invention disclosed herein.

For the purpose of describing the relative position of nucleotide sequences in a particular DNA molecule throughout this application, such as when a particular nucleotide sequence or residue is described as being situated “upstream,” “downstream,” “3′,” or “5′” relative to another sequence or residue, it is understood that it is the position of the sequences or residues in the “sense” or “coding” or “+” or “anti-template” strand of a DNA molecule that is being referred to.

The term “homology” as used herein refers to the percent identity between two polynucleotide or two polypeptide sequences and is identical with the term sequence identity as already provided hereinabove. Thus, two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

The practice of the present invention employs, unless otherwise expressly stated, conventional methods of virology, microbiology, molecular biology and recombinant DNA techniques within the skill of the art. Such techniques are explained fully in the literature. See, e.g., Sambrook, et al. Molecular Cloning: A Laboratory Manual (Current Edition); Cold Spring Harbor Press; Wu et al, Methods in Gene Biotechnology, (1997) CRC Press LLC, New York); Recombinant Gene Expression Protocols, Tuan, R.S. (Ed.) Methods in Molecular Biology, Vol. 62 (1997) Humana Press, Totowa, N.J.

All publications, patents and patent applications cited herein, whether supra or infra, are hereby incorporated by reference in their entirety.

The methods disclosed according to the present invention relate to the use of recombinant AAV vectors containing DNA sequences coding for heavy and light chains of antibodies specific for viruses, especially those causing respiratory disease, and including other microbes, such as bacteria, fungi and other parasites, most especially where the latter cause, or induce, or mediate, or otherwise aggravate, respiratory disease, or diseases, or irritations, or infections, otherwise related to the respiratory system. The methods of the present invention further relate to the use of such vectors for the in vitro and/or in vivo transduction of cells, such as respiratory and muscle cells, which can subsequently be introduced into a subject for treatment. The invention also provides for secretion of the produced protein in vivo, from transformed muscle cells, such that systemic delivery can be achieved, or from respiratory cells, for relatively direct action of the secreted protein, in this case a therapeutic immunoglobulin. Also in accordance with the present invention, the expressed protein is an antibody molecule, either single or double chain, especially said antibody is specific for an epitope found on a virus that induces respiratory disease, especially RSV, most especially the F or G epitope thereof.

In accordance with the present invention, rAAV expression vectors are constructed using known techniques to provide operably linked transcription facilitating components comprising a promoter sequence, the exogenous DNA coding for the antibody chain(s), and a terminating codon or region. The control elements are advantageously selected to be optimally functional in the mammalian cell of interest, especially a respiratory or muscle cell. The resulting construct which contains the operably linked components is bounded (5′ and 3′ ) with functional AAV inverted terminal repeat sequences. Suitable exogenous inserts will commonly be less than about 5 kilobases (kb) so that antibody protein sequences are well within the range of polypeptides that can be expressed by the transformed cells.

In carrying out the procedures of the present invention it is understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

Also in accordance with the methods of the present invention, administration of the recombinant vectors used to treat virus-induced respiratory diseases, especially diseases caused by RSV, comprises injecting a sample of said recombinant vector, especially recombinant AAV, or rAAV, into the tissues of a patient afflicted with such a respiratory disease, or at risk of contracting such disease. In one embodiment, the methods of the present invention comprise injection of a composition containing recombinant AAV into tissues such as muscle tissue or into lung tissue.

In another embodiment of the present invention, the recombinant AAV is administered to a patient afflicted with a virus-induced respiratory disease, or a patient at risk of contracting such a disease within a transformed cell. In this embodiment, a cell, especially a muscle or respiratory cell, such as a respiratory epithelial or connective tissue cell, is transformed, or otherwise transfected, with a polynucleotide, or polynucleotides, encoding polypeptide chains corresponding to the light and/or heavy chains of an antibody molecule, wherein said antibody molecule has affinity, and specificity, for one or more epitopes, but at least one epitope, of a virus that causes a respiratory disease.

In accordance with the present invention, said cells can be surgically excised from a patient afflicted with such a disease. Such excised cells are then transfected with the appropriate DNA contained within the genome of a vector as described herein, which encodes for the polypeptides of an antibody useful in treating or preventing said respiratory disease, such as RSV or PIV (parainfluenza virus), or otherwise transformed with said exogenous DNA, such as with the recombinant AAV particles described herein. The suitably transformed, or transfected, or recombinant, or genetically engineered cells so produced are then advantageously re-implanted back into the patient from whom they were originally excised and thereby permitted to express and secrete the encoded antibody molecule in sufficient amounts as to provide the appropriate treatment, or prophylactic action, as is the goal of the procedure.

In one embodiment of the present invention, the rAAV is engineered to contain exogenous DNA encoding one or more of the polypeptide chains of an antibody specific for, and having high affinity for, and high potency against, a virus causing the respiratory disease to be treated or prevented, especially where said virus is and most especially where the antibody is specifically directed against the F epitope of RSV.

In another embodiment of the present invention, the rAAV is engineered to contain exogenous DNA encoding one or more of the polypeptide chains of an antibody specific for, and having high affinity for, and high potency against the effects of chronic disease, such as asthma and the like. In specific embodiments, such antibodies include anti-interleukin 9 (anti-IL-9), which is useful in treating respiratory infections, as well as chronic diseases, such as asthma and other respiratory conditions and inflammations. The vectors disclosed herein may also contain DNA encoding other antibody molecules useful in the treatment of chronic infections, such as asthma, that specifically involve the respiratory system.

Antibodies encoded by genes contained in the rAAV vectors of the invention are also useful in the treatment of chronic diseases elsewhere in the body, such as the ophthalmic system, preferably disease conditions such as age related, especially diabetes related, macular degeneration and other retinopathies.

The present invention also relates to a recombinant AAV vector comprising exogenous DNA encoding at least one heavy or at least one light chain of an antibody specific for at least one epitope of RSV. In another embodiment, the present invention also relates to a recombinant AAV vector comprising exogenous DNA encoding at least one heavy and at least one light chain of an antibody specific for at least one epitope of RSV, so that exogenous DNA encoding both a heavy and a light chain polypeptide of an antiviral antibody is contained within the same recombinant AAV vector. Of course, in keeping with the present invention, the recombinant AAV disclosed herein may contain exogenous DNA coding for either of said heavy or light chains, in which case treatment and/or prevention of said virus-induced respiratory disease comprises administration to a patient afflicted therewith, or at risk thereof, of a therapeutically active amount of a mixture of said recombinant AAVs, preferably in the form of a composition containing said rAAVs suspended in a pharmacologically acceptable carrier, which includes any pharmaceutically suitable diluent or excipient.

In a most preferred embodiment, the recombinant AAV vector of the present invention contains an exogenous DNA encoding either a heavy chain, or a light chain, or both heavy and light chains, of an antibody disclosed in U.S. Pat. No. 5,824,307. In separate preferred embodiments, the rAAV vectors of the present invention contain polynucleotides encoding antibody polypeptide chains making up antibodies such as anti-IL-9, useful in treating RSV and other respiratory infections and chronic diseases, and Vitaxin, an anti-angiogenesis antibody, useful in the treatment of chronic ophthalmic conditions, especially conditions such as diabetic retinopathy and age related macular degeneration. Vitaxin is an αᵥβ₃-specific humanized monoclonal antibody well known in the art. [See: Wu et al, Proc. Natl. Acad. Sci., USA, 95:6037-6042 (May 1998)] For example, preparation and use of rAAV vectors containing coding sequences for Vitaxin II is described in Example 2, below. Treatment of ophthalmic conditions such as these can readily be effected by topical application of the vectors of the invention so as to transfer the appropriate gene sequences into cells, such as muscle cells, in and around the eye, as well as introduction of these vectors into more remote locations for ready transformation of the surrounding tissues and subsequent release of the encoded immunoglobulin molecules into the surrounding tissues for migration into the bloodstream and eventual arrival at ophthalmic locations.

In specific embodiments, the rAAV vectors of the present invention encode one or more polypeptide chains of antibodies designated CH1129, H1129, M1129, M1308F, H1308F, and MEDI-493, disclosed in U.S. Pat. No. 5,824,307 (the disclosure of which is hereby incorporated by reference in its entirety) and in Johnson et al, J. of Infectious Diseases,176,1215-1224(1997) (MEDI-493 disclosed).

Thus, in a specific embodiment the present invention relates to rAAV vectors comprising DNA encoding antibodies, such anti-IL-9 and anti-IL-9-receptors, which antibodies have the effect of prevent signal transduction between IL-9 and its respective receptor protein and thereby alleviating such chronic diseases as atopic asthma and the like. Use of such asthma associated factors as targets for treatment by non-antibody chemical agents is known in the art. [See: U.S. Pat. No. 5,908,839]. The methods of the present invention are also applicable to the treatment of non-asthmatic conditions, such as other types of allergy and allergic reactions.

In another embodiment, the antibodies encoded by the vectors disclosed herein find use against other respiratory conditions, such as bronchitis, bronchiolitis, pneumonia and cystic fibrosis, to name only a fraction. For example, it is known that there are genes coding for proteins of the calcium-activated chloride channel family that are induced by IL-9. Such a system thus provides a target for the antibody-encoding vectors disclosed herein, whereby such antibodies, when produced by cells transformed by the rAAV vectors of the present invention, serve as a therapeutic agent in this interleukin-9 mediated development of atopic allergy, asthma related disorders, and cystic fibrosis [also see International Publication WO 99/03488 and WO 97/08321], a list that is by no means exhaustive. For a further description of the operation of IL-9 in this scheme, see International Pat. No. WO 99/44620. New genes in the G-coupled protein receptor family are also induced by IL-9 and thereby provide an additional therapeutic target in IL-9 mediated development of atopic allergy and asthma-related disorders, as well as certain lymphomas and leukemias. [See: International Application WO 99/15656 for further description of this pathway]

In addition, various inflammations, such as inflammatory bowel diseases, have been shown to be Th₂ mediated in animals normally having a Th₁ mediated response [See: International Publication 98/27997]. Thus, up regulation of Th₂ has a palliative effect and could be aided by antibodies against Th₁, which can be supplied using rAAV vectors according to the present invention, such vectors being prepared in the same way as shown in the Examples below.

It is to be understood that all such uses are exemplary only and in no way limit the methods disclosed according to the present invention.

In addition, it has been shown [See: International Application WO 99/14242] one or more genes of the ras family is induced by IL-9. Thus, rAAV vectors according to the present invention can be used to deliver anti-IL-9 and anti-IL-9-receptor antibodies, as well as antibodies specific for the Ras protein, for treatment of atopic allergy, asthma and similar disorders, including certain leukemias and lymphomas. [See also International publications WO 98/24908]

The present invention also relates to a recombinant cell transformed with a recombinant AAV as described herein. Such a recombinant cell is advantageously selected from the group consisting of a muscle cell and a respiratory system cell.

In practicing the methods according to the present invention the selected nucleotide sequence, for example, the heavy or light chain of an antibody molecule, such as an antibody specific for IL-9 receptor, is operably linked to control elements that direct the transcription or expression thereof in the subject in vivo, such as within a muscle or respiratory cell, which control elements advantageously include those normally associated with the polypeptide to be expressed, especially any required signal sequences for the expressed protein, such as that required to direct secretion from the engineered cell. In addition, normal gene control sequences, such as endogenous or exogenous enhancer sequences are incorporated to amplify the expression of the desired gene sequences within the cells of interest. Useful heterologous control sequences generally include those derived from sequences encoding mammalian or viral genes, such as the SV40 early promoter, mouse mammary tumor virus LTR promoter, adenovirus major late promoter (Ad MLP), herpes simplex virus (HSV) promoter, cytomegalovirus (CMV) promoter (including CMV immediate early promoter region (CMVIE)), the Rous sarcoma virus (RSV) promoter. Control elements useful for expression of the polypeptides coding for antibodies within the methods of the present invention may also include wholly synthetic promoters, hybrid promoters, and the like. Sequences derived from nonviral genes, such as the murine metallothionein gene, also find use in the methods disclosed herein. Such promoter sequences are commercially available (for example, from Stratagene (San Diego, Calif.)).

For purposes of the present invention, for example, where the rAAV exogenous sequences, such as the sequence for antibody heavy chain, or light chain, or where polypeptides for both heavy and light chains are incorporated into the same vector, is to be expressed from muscle cells, the required control elements, such as muscle-specific and inducible promoters, enhancers and the like, are especially preferred. Such control elements include, but are not limited to, those derived from the actin and myosin gene families, such as from the myoD gene family (See: Weintraub et al. Science 251:761-766 (1991)), the myocyte-specific enhancer binding factor MEF-2 (Cserjesi and Olson Mol. Cell Biol. 11:4854-4862 (1991)), control elements derived from the human skeletal actin gene (Muscat et al. Mol. Cell Biol. 7:4089-4099 (1987)), the cardiac actin gene, muscle creatine kinase sequence elements (See: Johnson et al. Mol. Cell Biol. 9:3393-3399 (1989)) and the murine creatine kinase enhancer (mCK) element, control elements derived from the skeletal fast-twitch troponin C gene, the slow-twitch cardiac troponin C gene and the slow-twitch troponin I gene; hypoxia-inducible nuclear factors (Semenza et al. PNAS 88:5680-5684 (1991)), steroid-inducible elements and promoters (including the glucocorticoid response element (GRE) (See: Mader and White PNAS 90:5603-5607 (1993)), and other control elements.

Any such control elements can be tested for utility in controlling the exogenous genes of interest. These and other regulatory elements can be tested for potential in vivo efficacy using the in vitro myoblast model, which mimics quiescent in vivo muscle physiology, described in U.S. Pat. No. 5,858,351, the disclosure of which is hereby incorporated by reference in its entirety.

Similar considerations are important when respiratory or other cell types are used.

For the purposes of the present invention, suitable host cells for producing rAAV virions for therapeutic use include microorganisms, yeast cells, insect cells, and mammalian cells, that can be, or have been, used as recipients of an exogenous or heterologous DNA molecule, including all daughter cells derived from the cells so transfected. Numerous vectors have been described which encode Rep and/or Cap expression products for providing the aforementioned helper functions (for example, the vectors disclosed in U.S. Pat. No. 5,139,941 are advantageous).

Additionally, in vitro packaged rAAV vectors find use in the methods of the present invention because they permit the transformation of cells in vivo so as to effect the expression encoded protein products without requiring helper viruses.

Following recombinant AAV replication, the newly formed rAAV particles, containing the sequences encoding the exogenous antibody molecules, are readily purified from the host cell by any of a number of well known techniques, including dialysis of cell lyasate followed by chromatography to remove debris, or by using equilibrium centrifugation using CsCl gradients. The purified rAAV virions are then suspended in a pharmacologically acceptable carrier for use in the methods of the invention.

In accordance with the present invention, rAAV virions are conveniently introduced into a mammalian cell, such as a respiratory or a muscle cell, using either in vivo or in vitro transducing techniques (i.e., techniques for introducing a vector into the cell of interest). For example, if muscle cells are to be transduced in vitro, the desired recipient muscle cell will be removed from the subject, transduced with rAAV virions and reintroduced into the subject using standard methodology. Alternatively, syngeneic or xenogeneic muscle cells can be used where those cells will not generate an inappropriate immune response in the subject.

For in vivo delivery, the rAAV virions are formulated into pharmaceutical compositions and administered by any of a number of well known techniques, such as by injection directly into skeletal muscle, or applied topically to respiratory tissue or to ophthalmic tissue, depending on the condition to be treated or prevented, or even given intravenously.

For in vivo delivery specifically into respiratory tissue, the rAAV virions, suitably suspended in a pharmacologically acceptable carrier, diluent or excipient, can be introduced into the respiratory system using any convenient form of suspension, including a powder, suspension, spray, or the like. Appropriate devices for introducing such suspensions into the respiratory system or tract are well known in the art and need not be reviewed here.

In accordance with the methods of the present invention, pharmaceutical compositions will comprise sufficient genetic material contained in the rAAV vectors to produce a therapeutically effective amount of the protein of interest, i.e., an amount of therapeutic immunoglobulin, such as one or more of the neutralizing antibodies useful in applying the methodology disclosed herein and in sufficient quantity to reduce the amount of infecting microbe, such as a virus, for example, RSV, PIV, and the like, or some other microbe, such as bacteria, fungi, and parasites, especially where such microbes infect the respiratory system. The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J. current edition).

Appropriate doses will depend on the age of the recipient. For example, in the case of antibodies directed against respiratory syncytial virus (RSV), this may be a child or an elderly person. Such considerations also involve the mode of administration, such as by implanting transduced cells, or in situ delivery of the rAAV vectors, such as by an aerosol spray. Other factors include the severity and progress of the infection. An appropriate effective amount can be readily determined by one of skill in the art. For example, in the use of some of the most potent and highly specific antibodies used to treat respiratory conditions, including viral infections and chronic conditions, including asthma and allergies, the maximal potency, or biological activity, has been observed at doses of about several mg/kg. For example, about 15 mg/kg body weight in the case of RSV infections. Notably, even as little as 0.1 mg/ml of serum is sufficient to combat RSV so that doses providing equivalent final concentrations are in general sufficient. For example, high level secretion of recombinant protein by AAV-tranduced cells in mice (up to 1 mg/ml) has been shown by Song et al, Proc Natl Acad Sci USA 95:14384-14388 (1998). Thus, 0.1 mg/ml, useful in treating viruses such as RSV, is sufficient for therapeutic purposes in accordance with the methods disclosed herein.

In accordance with the present invention, what constitutes a therapeutically effective amount often falls in a relatively broad range that is best determined through clinical trials like those required for FDA approval. For example, for in vivo injection directly to skeletal muscle, a therapeutically effective dose can range from about 10⁶ to about 10¹⁵ recombinant AAV particles, with at least about 10¹⁰ rAAV particles being preferred.

A lower dose may be useful for in vitro transduction or transformation of cells and the transduced or transformed cells are introduced into the patient as opposed to direct administration of rAAV vectors. Other effective dosages can be readily established by one of ordinary skill in the art through routine trials establishing common dose response curves.

Dosage treatment may be a single dose schedule or a multiple dose regimen, with as many doses as necessary being provided. One of skill in the art can readily determine an appropriate number of such doses. Such formulations and dose schedules are not intended to limit the scope of the present invention in any way.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

### EXAMPLE 1

This example shows generation of an AAV transfer vector for expression of palivizumab heavy and light chain genes.

For expression of palivizumab, an IgG immunoglobulin (useful in treating respiratory diseases caused by viruses (See: Pediatrics, 102 (3 Pt. 1), pp. 531-537 (1998)), such as RSV and PIV and whose sequence is described as Medi-493 in Johnson et al, J. of Infectious Diseases, 176, 1215-1224 (1997) and as H1129 in U.S. Pat. No. 5,824,307 (it is a commercially available antibody), the disclosures of both of which are hereby incorporated by reference in their entirety], the heavy and light chain genes are cloned into the vector pTR-UF12.1, containing the ITRs of AAV-2, enabling expression of both genes using a single promoter. The first gene, the palivizumab heavy chain gene, is cloned immediately downstream of the promoter region using HindIII and EcoRV restriction sites in the vector. The resulting vector, a recombinant AAV, is called pTR-SyHC. Next the palivizumab light chain is inserted into pTR-SyHC as a NotI to SalI fragment. This places the light chain gene after the heavy chain segment and immediately downstream from an internal translation reinitiation sequence from encephalomyocarditis virus. This vector is called pTR-SyHL. Alternatively, a separate construct can be prepared in which the same fragment is inserted into pTR-UF12.1, generating the vector pTR-SyLC. Thus expression of heavy and light chains can be accomplished with either a single construct, pTR-SyLH, or by co-tranfecting pTR-SyH and pTR-SyL. Here, the cell line known as 293 is used for transfection of pTR-SyLH (or co-transfection with pTR-SyH and pTR-SyL). Here, superinfection with the vector d27.1-rc (Conway et al. Gene Therapy 6:986-983), derived from an HSV-1 ICP deletion mutant and containing the rep and cap genes of AAV-2, supplies the appropriate helper functions, thereby accomplishing the required rescue of AAV-palivizumab IgG. Purification is achieved by filtration of a lysate of 293 cells to remove rHSV particles, followed by ion exchange and affinity chromatography to remove cellular impurities (as has been described in Zolotukhin et al; Gene Therapy 6:973-985 (1999)).

For use in therapy, recombinant AAV-palivizumab particles (>10¹⁰) are delivered to human subjects by direct intramuscular injection into skeletal muscle. Alternatively, systemic infusion to infect hepatocytes and other tissues, or inhalation to infect cells within the lung, accomplishes the same result. Upon infection with rAAV-palivizumab, the IgG molecules are manufactured, assembled, and secreted.

### EXAMPLE 2

This example shows generation of an AAV transfer vector for expression of Vitaxin-II heavy and light chain genes.

For expression of the Vitaxin-II IgG, the heavy and light chain genes are cloned into the vector pTR-UF12.1, containing the ITRs of AAV-2, enabling expression of two genes from a single promoter. The first gene, the Vitaxin-II heavy chain gene, is thereby cloned immediately downstream of the promoter region using the Hind III and EcoRV sites in the vector. The resulting rAAV vector is called pTR-ViHC. Next the Vitaxin-II light chain are inserted into pTR-ViHC as a NotI to Sa/I fragment. This places the light chain gene after the heavy chain segment and immediately downstream from an internal translation reinitiation sequence derived from encephalomyocarditis virus. This rAAV vector is called pTR-ViHL. Alternatively, a separate construct can be prepared in which the same fragment is inserted into pTR-UF12.1, generating the vector pTR-ViLC. Thus expression of heavy and light chains can be accomplished with either a single construct, pTR-ViLH, or by co-tranfecting pTR-ViH and pTR-ViL, as in Example 1, above. Here, the 293 cell line is used for transfection of pTR-ViLH or co-transfection with pTR-ViH and pTR-ViL. Superinfection with the vector d27.1-rc (Conway et al. Gene Therapy 6:986-983), derived from an HSV-1 ICP deletion mutant which contains the rep and cap genes of AAV-2, supplies the appropriate helper functions, will result in the rescue of AAV-Vitaxin II IgG. Purification is achieved by filtration of a lysate of 293 cells to remove rHSV particles and followed by ion exchange and affinity chromatography to remove cellular impurities as described (Zolotukhin et al; Gene Therapy 6:973-985 (1999)).

For therapeutic use, recombinant AAV-Vitaxin IgG particles (>10¹⁰) are delivered to human subjects by direct intramuscular injection. Alternatively, systemic infusion to infect hepatocytes and other tissues, or by inhalation to infect cells within the lung, or by intraocular injection to infect the retina or other cells within the eye, accomplish the desired therapeutic effect. Upon infection with rAAV-Vitaxin II IgG, the antibody molecules are manufactured, assembled, and secreted.

What is claimed is:

1. A recombinant adeno-associated virus (rAAV) vector whose genome comprises one or more polynucleotide sequences encoding at least one polypeptide of an antibody wherein said polynucleotide is operably linked to control elements that direct intracellular transcription and translation of said polynucleotide when said rAAV is inserted into a mammalian cell.

2. The vector of claim 1 wherein said polypeptide is the light chain of said antibody.

3. The vector of claim 1 wherein said polypeptide is the heavy chain of said antibody.

4. The vector of claim 1 wherein said polynucleotide encodes both a heavy and a light chain of said antibody.

5. The vector of claim 1 wherein said antibody is specific for at least one epitope found on a microbial organism.

6. The vector of claim 5 wherein said microbe is selected from the group consisting of viruses, bacteria, fungi and parasites.

7. The vector of claim 6 wherein said microbe is a virus.

8. The vector of claim 7 wherein said virus is selected from the group consisting of respiratory syncytial virus (RSV) and parainfluenza virus (PIV).

9. The vector of claim 5 wherein said microbial organism causes or induces a disease or inflammation of the respiratory system.

10. The vector of claim 9 wherein the disease is selected from the group consisting of bronchitis, bronchiolitis and pneumonia.

11. The vector of claim 5 wherein said antibody is selected from the group consisting of CH1129, H1129, M1129, M1308F, H1308F, and MEDI493.

12. The vector of claim 1 wherein said antibody is an antibody involved in mediating a non-infectious disease of the respiratory system.

13. The vector of claim 12 wherein said disease is selected from the group consisting of asthma, allergies, and cystic fibrosis.

14. The vector of claim 1 wherein said antibody is selected from the group consisting of anti-interleukin-9 (anti-IL-9), anti-interleukin-9-receptor (anti-IL-9-r), anti-Ras protein and vitaxin II.

15. The vector of claim 1 wherein said antibody is an antibody having a therapeutic effect on diseases or inflammations of the ophthalmic system.

16. A composition comprising a therapeutic amount of the recombinant vectors of claim 1 suspended in a pharmacologically acceptable carrier.

17. A method of treating a disease in a patient afflicted therewith comprising administering to said patient a therapeutic amount of the composition of claim 16.

18. The method of claim 17 wherein said administration comprises injecting a sample of said recombinant AAV into the tissues of said patient.

19. The method of claim 18 wherein said tissues are selected from the group consisting of respiratory tissue, muscle tissue, and ophthalmic tissue.

20. The method of claim 17 wherein said disease is selected from the group consisting of viral infection, asthma, allergy, and macular degeneration.

21. A recombinant cell whose genome comprises one or more rAAV vectors of claim 1.

22. The recombinant cell of claim 21 wherein said cell produces the polypeptide encoded by the polynucleotide contained in the genome of said rAAV particles.

23. The recombinant cell of claim 22 wherein said cell is selected from the group consisting of muscle cells, respiratory cells, ophthalmic cells.

24. A method of treating a respiratory disease in a patient comprising administering to said patient a therapeutic amount of the recombinant cells of claim 22.
